(19) United States
(12) Patent Application Publication
(10) Pub. No.: US 2005/0186281 A1
(43) Pub. Date: Aug. 25, 2005

(54) Antimicrobial sanitizing composition

(75) Inventors: Robert Blaine Lalum, Antelope, CA (US); Asia Kogan, Citrus Heights, CA (US); Zhong Wei Xie, Folsom, CA (US); Yu Yao, Rocklin, CA (US); Gary Linn Robinson, Livermore, CA (US)

Correspondence Address: T. Ling Chwang, Suite 600, 2435 N. Central Expressway, Richardson, TX, 75080, US

(73) Assignee: Mionix Corporation, Rocklin, CA (US)

(21) Appl. No.: 10/948,762

(22) Filed: Sep. 23, 2004

Related U.S. Application Data
(60) Provisional application No. 60/506,659, filed on Sep. 26, 2003.

(51) Int. Cl. A61K 31/715; A61K 31/14; A61K 9/14

(52) U.S. Cl. 424/487; 514/643; 514/54

(57) ABSTRACT

A surfactant-free, alcohol-free, water based antimicrobial composition comprising a dissolved phenolic antimicrobial agent, such as chloroxylenol (“PCMX”), and with a pH range between 5.5 to 6.5. The dissolved phenolic antimicrobial agent is able to achieve a high level of antimicrobial activity despite being present at a low percent concentration. Other ingredients include a glycol, glycerin, a neutralizing agent, a preservative, an acrylic polymer, a polysaccharide polymer, and deionized water. Optional ingredients include fragrance, additional moisturizers, and a pH adjuster. The process for formulating the antimicrobial composition utilizes an anhydrous water soluble solvent complex to dissolve the phenolic antimicrobial agent.

This application claims priority to U.S. Provisional Patent Application Ser. No. 60/506,659, entitled “Antimicrobial Sanitizing Composition” filed on Sep. 26, 2003, the entire content of which is hereby incorporated by reference.

### BACKGROUND

This invention relates to a surfactant-free, alcohol-free, water based antimicrobial composition containing a dissolved phenolic antimicrobial agent in a water phase, and the method by which it is produced.

Topical antimicrobial compositions, such as soaps and washes, are frequently used to minimize residual microbial presence on skin surfaces and provide protection from future contamination. These antimicrobial compositions typically contain one or more antimicrobial agents.

Antimicrobial agents are chemicals that kill or inhibit the growth of microbial organisms. Examples of antimicrobial agents include bisbiquanide, diphenyl compounds, benzyl alcohols, trihalocarbanilides, quaternary ammonium compounds, ethoxylated phenols, alcohols, cationic surfactants and phenolic compounds. Phenolic antimicrobial agents such as para-chloro-meta-xylenol (“PCMX”) kill microbial organisms by cell wall disruption and enzyme inactivation.

However, phenols have extremely low solubility in water. Typically, solubility of the phenolic antimicrobial agent is increased through the addition of surfactants. U.S. Pat. No. 6,451,748 to Taylor et al. discloses an antibacterial composition containing a phenolic antimicrobial agent that is solubilized in a surfactant. Surfactants in water form micelles around the phenolic antimicrobial agent. These micelles allow dispersion of the agent in water, but also prevent the agent from immediately contacting the microbial organisms.

One example of a phenolic antimicrobial agent is para-chloro-meta-xylenol (“PCMX”). PCMX is a desirable antimicrobial agent and is particularly effective against a wide variety of Gram positive and Gram negative bacteria, some viruses, and fungi. PCMX has a phenolic chemical structure and is related to compounds such as cresol, carbolic acid, and hexachloroprene. PCMX goes by a variety of other names, including chloroxylenol; 4-chloro-3,5 xylenol; 4-chloro-3,5-dimethylphenol; 2-chloro-m-xylenol; 2-chloro-5-hydroxy-m-xylene; 2-chloro-5-hydroxy-m-xylene; 2-chloro-5-hydroxy-1,3-dimethylbenzene; 4-chlor-1-hydroxy-3,5-dimethyl benzene; and 3,5-dimethyl-4-chlorophenol. Antimicrobial formulations containing phenolic agents such as PCMX as disinfecting ingredients are known in the art and disclosed by Garabedian, et al., U.S. Pat. No. 4,632,772; Corti, et al., U.S. Pat. No. 5,114,978; Kahn, et al., U.S. Pat. No. 5,439,681; Woodin, Jr., et al., U.S. Pat. No. 5,494,533; Fendler, et al., U.S. Pat. No. 5,635,462; Beerse, et al., U.S. Pat. No. 6,287,577; Childers, et al., U.S. Pat. No. 6,413,921; Sine, et al., U.S. Pat. No. 6,423,329; Stack, U.S. Pat. No. 6,517,854; and Asmus, et al., U.S. Pat. No. 6,582,711.

Although the antimicrobial effectiveness of PCMX is desirable, formulations containing PCMX are difficult to prepare due to its low solubility and incompatibility with many surfactants as well as other types of compounds. The efficacy of PCMX is often compromised by a variety of factors, such as additional ingredients (e.g., surfactants), pH level, and solubility. For example, see Kahn et al., U.S. Pat. No. 5,439,681, which discloses the difficulty of combining surfactants with PCMX while preserving antiseptic efficacy. Dryness and irritation to the skin are also frequently associated with phenolic antimicrobial compounds such as PCMX.

Traditionally, two methods have been used to solubilize or dissolve PCMX. The first method solubilizes PCMX using high levels of surfactant, sometimes in the presence of an alcohol. This method is typically used in antimicrobial soaps. Due to the high level of surfactant, these products must be washed off of the skin or severe irritation will result. Once washed off, the skin is unprotected and susceptible to recontamination by microbes. The second method dissolves PCMX using high levels of volatile organic solvents such as alcohol. The volatile organic solvent may be present between 60% and 70%. This high level of organic solvent results in undesirable odors as well as dry and irritated skin. Furthermore, once the solvent evaporates, the activity of the PCMX may be reduced. Existing antimicrobial formulations using phenolic antimicrobial agents suffer from the fact that increased use of various surfactants and lipid-restoring compounds, which are used to solubilize or dissolve the phenolic antimicrobial active ingredient, reduce the antimicrobial effectiveness of the formulation.

### SUMMARY

This invention is directed to an antimicrobial composition in the form of a surfactant-free and alcohol-free water based product with a pH of 5.5-6.5 and the method by which it is produced. The antimicrobial formulation is a topical “waterless” sanitizing or disinfecting composition that contains a dissolved phenolic antimicrobial agent such as p-chloro-m-xylenol (“PCMX”). Even at very low levels of dissolved PCMX, the antimicrobial composition demonstrates very high antimicrobial activity, with a significant and immediate reduction in the number of microbes present on contact.

The antimicrobial sanitizing composition of the current invention is preferably a hand sanitizing composition. The antimicrobial composition has a low level of antimicrobial agent, such as PCMX, but a high level of antimicrobial activity. The antimicrobial effect of the antimicrobial composition is immediate. The antimicrobial composition contains an active amount of a phenolic antimicrobial agent such as PCMX, but is alcohol-free, surfactant-free, and contains the antimicrobial agent in the continuous water phase. The antimicrobial composition also demonstrates improved antimicrobial effectiveness when compared to other commercially available antimicrobial sanitizing products.

The current invention also pertains to a method of using an anhydrous solvent complex to completely dissolving the phenolic antimicrobial agent under a pH of about 9.0 (in a 10% water solution). The anhydrous solvent complex contains a diol, a triol, an amine, and mixtures thereof. The dissolved phenolic antimicrobial agent maintains a high level of efficacy, despite temperature fluctuations, until it is delivered and released on the skin surface.

The antimicrobial sanitizing composition is non-toxic, non-flammable, non-staining, and milder to the skin than many currently available products. Other antimicrobial products may use alcohol, iodine, or additional additives which break down the skin's natural water barrier. The current antimicrobial composition combines a highly effective and fully dissolved phenolic antimicrobial agent with conditioners, emollients, and botanicals which moisturize the skin and allow the composition to be used repeatedly as a routine cleanser.

The antimicrobial sanitizing composition contains a mixture of several components. The bulk of the antimicrobial composition is made up of deionized water. The water is present as fixed-water bulk. The active ingredient in the sanitizing composition is a phenolic antimicrobial agent, such as PCMX. Other ingredients include solvents, moisturizers, and pH adjusters such as glycerin, propylene glycol, and triethanolamine (“TEA”). A further ingredient is a preservative, such as phenoxyethanol. Other ingredients are an acrylic polymer, such as a carbomer, and a polysaccharide polymer, such as xantham gum. Additional optional ingredients include fragrances and additional moisturizers, such as vitamin E succinate and aloe vera gel. An additional pH adjuster may be added, such as acidic calcium sulfate (“ACS”).

The antimicrobial sanitizing composition does not contain a surfactant, nor does it contain a volatile organic solvent, such as alcohol.

The antimicrobial agent PCMX is highly insoluble in water. However, PCMX must be dissolved in order to be active. A dissolved phenolic antimicrobial agent, such as dissolved PCMX, is one that has been essentially dissolved in a solvent or a mixture of solvents with only a very fine suspension of crystals, if any. If excessive crystals or precipitate are observed, the PCMX is in a crystal state and is not antimicrobially active. Thus, another aspect of the invention relates to a method for dissolving a phenolic antibacterial agent such as PCMX. The method utilizes a water soluble anhydrous solvent complex that can dissolve PCMX, particularly at low levels of about 0.1% to about 0.5%, in concentrations of water greater than 90%. The water soluble anhydrous solvent complex comprises a diol, a triol, an amine, and mixtures thereof. The water soluble anhydrous solvent complex preferably comprises a mixture of glycerin, propylene glycol, and TEA. PCMX is mixed into the solvent complex, with optional heating, until it is completely dissolved.

Another aspect of the invention is the formation of the antimicrobial composition. As discussed above, the PCMX is dissolved in a water soluble anhydrous solvent complex. Other components of the antimicrobial composition, namely the acrylic polymer, the polysaccharide polymer, and the preservative, are mixed in a separate container to form a polymer dispersion. The dissolved PCMX is slowly mixed into the polymer dispersion so that the dispersion of the PCMX into the forming gel occurs simultaneously with water fixation. Fixation occurs when the acrylic polymer polymerizes after being neutralized with a suitable neutralizing agent, such as TEA. Water fixation occurs when water is fixed into the matrix of the thickened polymer as it is formed. This method of incorporating the PCMX into the polymer dispersion does not result in precipitation of the PCMX. Thus, the PCMX, even in a small amount, remains highly active and antimicrobially effective.

The three components useful for dissolving PCMX have several different functions in the formulation. TEA keeps the pH of the solvent mixture at a pH of 9.0 (in a 10% water solution) before it is added to the polymer dispersion. The pH of glycerin and propylene glycol is normally around 7.0. TEA also neutralizes the acrylic polymer to initiate polymerization and facilitates thickening of the polymer to an appropriate viscosity, which is dependent on pH. For a more viscous solution, 0.45% TEA can be added to achieve a final pH of 7.0. Propylene glycol and glycerin serve two separate functions. They are solvents for PCMX and moisturizers for the skin.

The water-soluble anhydrous solvent complex can dissolve both high and low concentrations of PCMX. The current antimicrobial sanitizing composition preferably utilizes low levels of PCMX in order to minimize skin irritation.

PCMX has antimicrobial activity when it is present in a solution having a pH of between about 4.0 and 9.0. High levels of antimicrobial activity have typically been reached between about pH 4.0 and 9.0, with higher activity usually seen at pH 9.0. Thus, the majority of PCMX-based soaps and disinfectants have a pH around 9.0. These pH extremes can be irritating to the skin. The pH of the antimicrobial composition is adjusted to be between 5.5 and 6.5, which is more closely balanced with the pH that occurs naturally on the surface of the skin and is less irritating. Furthermore, the present composition does not lose antimicrobial effectiveness at its formulated pH range.

The antimicrobial composition has an exceptionally high broad spectrum antibacterial efficacy, as measured by a rapid kill of bacteria. The antimicrobial composition inhibits the growth of harmful microorganisms such as Salmonella choleraesuis, Pseudomonous aeruginosa, Staphylococcus aureus, and Escherichia coli. Antimicrobial activity is typically correlated with a log scale or percent reduction in microbial populations resulting from contact with the antimicrobial formulation. After contact times generally ranging from 15 seconds to 5 minutes, a log reduction of 3-5 is most preferred, a log reduction of 1-3 is less preferred, and a log reduction of less than 1 is acceptable. The current antimicrobial composition exhibits a log reduction of at least 5-6 against Gram positive bacteria (e.g. Staphylococcus aureus) and Gram negative bacteria (e.g., Escherichia coli) after only 30 seconds of contact.

The antimicrobial sanitizing composition is used without water and remains on the skin after drying. The antimicrobial composition may be used in consumer products such as skin cleanser, “waterless” hand sanitizing gel, and the like.

### DETAILED DESCRIPTION OF PREFERRED EMBODIMENTS

The antimicrobial sanitizing composition is surfactant-free, alcohol-free, water based, and contains a low percentage of dissolved phenolic antimicrobial agent. The phenolic antimicrobial agent is dissolved and present in a continuous water phase. The antimicrobial composition has a pH between about 5.5 and about 6.5 and shows high antimicrobial activity against a wide spectrum of organisms after very short contact times. A preferred embodiment of the antimicrobial sanitizing composition comprises a dissolved phenolic antimicrobial agent, a glycol, glycerin, a neutralizing agent, a preservative, an acrylic polymer, a polysaccharide polymer, and deionized water. Optional additional ingredients are preferably a fragrance, additional moisturizers, and a pH adjuster. The percentages given here are weight percent (by weight).

The antimicrobial sanitizing composition contains a dissolved phenolic antimicrobial agent. The phenolic antimicrobial agent may be PCMX, 2,4,4′-trichloro-2′-hydroxy-diphenylether, benzylalkonium chloride, or 4-chloro-3,5-dimethylphenol. Preferably, the phenolic antimicrobial agent is PCMX. The phenolic antimicrobial agent may be present in the antimicrobial composition from about 0.1% to about 3.75% (by weight), preferably from about 0.1% to about 0.75%, more preferably from about 0.1% to about 0.5%, and most preferably at about 0.3%.

In addition, a preferred embodiment of the antimicrobial composition contains a glycol. The glycol may be propylene glycol, hexylene glycol, triethylene glycol, ethylene glycol, or diethylene glycol. Preferably, the glycol is propylene glycol. The antimicrobial composition may contain from about 1% to about 8% glycol, preferably from about 1% to about 5%, more preferably from about 1% to about 4%, and most preferably at about 2%.

Another preferred embodiment of the antimicrobial composition contains glycerin as a solvent and moisturizer. The antimicrobial composition may contain from about 2% to about 10% glycerin, preferably from about 2% to about 7%, more preferably from about 2% to about 5%, and most preferably at about 3%. As a solvent, glycerin is most preferred. In alternative embodiments, other additional moisturizers may be substituted for glycerin. These moisturizers include polyols such as sorbitol, mannitol, maltitol, isomalt, xylitol, and erythritol.

A preferred embodiment of the antimicrobial composition also includes a neutralizing agent. The neutralizing agent may be an organic base such as TEA, or an inorganic base such as 1M sodium hydroxide or 1M potassium hydroxide. Preferably, the neutralizing agent is TEA at a strength of about 99%. In some embodiments, TEA acts as both a solvent and a neutralizing agent. In alternative embodiments, the neutralizing agent is a separate compound and may be ammonium hydroxide, polar organic amines such as diethylamine, diisopropanolamine, triethylamine and triethanolamine, low polarity amines such as cocamine (Armeen® CD, Akzo Nobel, Stenungsund, Sweden) and cocamine substituted with polyoxyethylene units (Ethomeen® C/12, Akzo Nobel), or amino acids such as glycine, 3-alanine, and 6-amino hexanoic acid. The neutralizing agent may be present from about 0.1% to about 1%, preferably from about 0.1% to about 0.5%, more preferably from about 0.1% to about 0.4%, and most preferably at about 0.15%.

A preferred embodiment of the antimicrobial composition also includes a preservative. The preservative may be phenoxyethanol, chlorphenesin, iodopropynyl butylcarbamate, benzoic acid, potassium sorbate, or sorbic acid. Preferably, the preservative is phenoxyethanol, or a phenoxyethanol-based preservative. In alternate embodiments, a mixture of iodine carbamate is included in the phenoxyethanol solvent base. The preservative may be present in the antimicrobial composition from about 0.1% to about 1%, preferably from about 0.3% to about 0.7%, and more preferably from about 0.45% to about 0.6%.

An additional preferred embodiment contains about 0.1% to about 1% by weight of an acrylic polymer, such as a carbomer, preferably from about 0.1% to about 0.5%, more preferably about 0.1% to about 0.3%, and most preferably at about 0.25%.

Another preferred embodiment of the antimicrobial composition contains a polysaccharide polymer. The polysaccharide polymer may be xantham gum, methylcellulose, propylcellulose, acacia gum, arabic gum, gum ghatti, guar gum, benzoin gum, tamarind gum, karaya gum, or gum accroides. Preferably, the polysaccharide polymer is xantham gum. The polysaccharide polymer may be present from about 0.02% to about 0.5% by weight, preferably about 0.02% to about 0.25%, more preferably from about 0.02% to about 0.075%, and most preferably at about 0.05%.

The antimicrobial composition also contains deionized water in an amount to bring the mixture up to 100%.

An optional ingredient in additional preferred embodiments is a fragrance, which may be present from about 0.05% to about 0.5%, preferably about 0.05% to about 0.2%, and more preferably about 0.05% to about 0.1%. Any type of natural or artificial fragrance may be used.

Other optional ingredients are additional moisturizers, such as vitamin E and aloe vera. Vitamin E may be present as vitamin E succinate, vitamin E acetate, or vitamin E (alpha tocopherol). Preferably, vitamin E succinate is used. The vitamin E may be present from about 0.005% to about 0.4%, preferably from about 0.005% to about 0.2%, more preferably from about 0.005% to about 0.07%, and most preferably at about 0.01%. Aloe vera may be present as a gel or extract, preferably a gel, from about 0.025% to about 1%, preferably from about 0.025% to about 0.5%, more preferably from about 0.025% to about 0.1%, and most preferably at about 0.05%.

An additional optional ingredient in a preferred embodiment is a pH adjuster such as acidic calcium sulfate (“ACS”), one or more organic acids, such as lactic acid, glycolic acid, citric acid, or any of the alpha hydroxy acids, or one or more inorganic acids, such as sulfuric or phosphoric acid.

ACS may also be defined as an acidic, or low pH, solution of sparingly-soluble Group IIA-complexes (“AGIIS”) (See, U.S. patent application Ser. No. 09/500,473, “Acidic Solution of Sparingly-Soluble Group IIA Complex”; see also, U.S. Pat. No. 6,436,891, “Adduct Having An Acidic Solution of Sparingly-Soluble Group IIA Complexes”; the entire content of each of the two is hereby incorporated by reference). The term “complex,” as used herein, denotes a composition wherein individual constituents are associated. “Associated” means constituents are bound to one another either covalently or non-covalently, the latter as a result of hydrogen bonding or other inter-molecular forces. The constituents may be present in ionic, non-ionic, hydrated or other forms.

The acidic solution of sparingly-soluble Group IIA-complex salt (“AGIIS”) can be prepared in several ways. Some of the methods involve the use of Group IA hydroxide but some of syntheses are devoid of the use of any added Group IA hydroxide, although it is possible that a small amount of Group IA metal may be present as “impurities.” The preferred way of manufacturing AGIIS (or ACS) is not to add Group IA hydroxide to the mixture. As the phrase implies, AGIIS is highly acidic, ionic, with a pH of below about 2.

The preferred method of preparing AGIIS (or ACS) involves mixing a mineral acid with a Group IIA hydroxide, or with a Group IIA salt of a dibasic acid, or with a mixture of the two Group IIA materials. In the mixing, a salt of Group IIA is also formed. Preferably, the starting Group IIA material or materials selected will give rise to, and form, the Group IIA salt or salts that are sparingly soluble in water. The preferred mineral acid is sulfuric acid, the preferred Group IIA hydroxide is calcium hydroxide, and the prefer Group IIA salt of a dibasic acid is calcium sulfate. Other examples of Group IIA salt include calcium oxide, calcium carbonate, and “calcium bicarbonate.”

AGIIS (or ACS) is preferably prepared by mixing calcium hydroxide with concentrated sulfuric acid, with or without an optional Group IIA salt of a dibasic acid (such as calcium sulfate) added to the sulfuric acid. For every mole of concentrated acid, such as sulfuric acid, the amount, in moles, of calcium hydroxide used is application specific and ranges from about 0.1 to about 1. The optional calcium sulfate can be added to the concentrated sulfuric acid prior to the introduction of calcium hydroxide into the blending mixture. The addition of calcium sulfate to the concentrated sulfuric acid appears to reduce the amount of calcium hydroxide needed for the preparation of AGIIS (or ACS). For every mole of concentrated acid, such as sulfuric acid, the amount, in moles, of calcium carbonate ranges from about 0.001 to about 0.2, depending on the amount of calcium hydroxide used. Other optional reactants include calcium carbonate and gaseous carbon dioxide being bubbled into the mixture. Regardless of the use of any optional reactants, the use of calcium hydroxide is desirable.

The following procedure may be used to make 1.2-1.5 N AGIIS (or ACS). An amount of 1055 ml (19.2 moles, after purity adjustment and taking into account the amount of acid neutralized by base) of concentrated sulfuric acid (FCC Grade, 95-98% purity) is slowly added with stirring, to 16.868 L of RO/DI water in each of reaction flasks a, b, c, e, and f. The amount of water is adjusted to allow for the volume of acid and the calcium hydroxide slurry. The mixture in each flask is mixed thoroughly. Each of the reaction flasks is chilled in an ice bath until the temperature of the mixture in the reaction flask is about 8-12° C. The mixture is continuously stirred at a rate of about 700 rpm.

Separately, a slurry is made by adding RO/DI water to 4 kg of calcium hydroxide (FCC Grace, 98% purity) making a final volume of 8 L. The mole ratio of calcium hydroxide to concentrated sulfuric acid is 0.45 to 1. The slurry is a 50% (W/V) mixture of calcium hydroxide in water. The slurry is mixed well with a high-shear-force mixer until the slurry appears uniform. The slurry is then chilled to about 8-12° C. in an ice bath and continuously stirred at about 700 rpm.

To each of the reaction flasks is added 150 ml of the calcium hydroxide slurry every 20 minutes until 1.276 L (i.e. 638 g dry weight, 8.61 moles, of calcium hydroxide) of the slurry has been added to each reaction vessel. The addition is again accompanied by mixing at about 700 rpm. After the completion of the addition of the calcium hydroxide to the reaction mixture in each reaction vessel, the mixture is filtered through a 5-micron filter. The filtrate is allowed to sit for 12 hours, then the clear solution is decanted to discard any precipitate formed. The resulting product is AGIIS (or ACS) having an acid normality of 1.2-1.5.

The pH adjuster of the antimicrobial composition is preferably ACS at a normality of about 0.15N. When added to the antimicrobial composition, the pH adjuster is preferably present at about 0.01% to about 0.05% by weight, preferably from about 0.02% to about 0.04%, and more preferably about 0.03%.

The final pH of the antimicrobial composition ranges from about 5.5 to about 6.5.

An additional preferred embodiment is a method for preparing the antimicrobial composition. To prepare the antimicrobial composition, a blend of the acrylic polymer and the polysaccharide polymer is combined with the water bulk, and then the preservative is added to polymer/water mixture. These components are mixed until the polymers and preservative are completely dispersed within a water phase. The water phase is then mixed for about 30 to about 40 minutes under sufficiently rigorous conditions to yield a homogeneously blended polymer dispersion having a pH of about 3.0 to 3.5. To this homogeneously blended mixture is optionally added fragrance, additional moisturizers, and a pH adjuster. The components are mixed to form a homogeneous blend at room temperature.

A further preferred embodiment is a method for dissolving the phenolic antimicrobial agent, such as PCMX. In order to completely dissolve the phenolic agent for use in the antimicrobial composition, a solvent mixture of a glycol, glycerin, and a neutralizing agent is first prepared in a separate container. The phenolic antimicrobial agent is then added to the solvent mixture and mixed until the agent is completely dissolved. Optionally, this mixture may be heated to and maintained at a temperature from about 35° C. to about 50° C., preferably at about 40° C. The mixture is mixed at a speed ranging from about 200 rpm to about 500 rpm, and preferably at about 300 rpm. The mixing is performed for about 20 to 30 minutes to produce a homogeneous clear solution.

The mixture containing the dissolved phenolic agent is then added to the first blended mixture and the two solutions are mixed for about 20 to about 30 minutes to produce a homogeneously blended antimicrobial composition.

The antimicrobial sanitizing composition is capable of producing a log reduction of Gram positive and Gram negative bacteria of 5 to 6 after about 30 seconds of contact, as measured against Staphylococcus aureus, Salmonella choleraesuis, Pseudomonas aeroginosa, and Escherichia coli. The antimicrobial composition demonstrates a high level of antimicrobial efficacy throughout its pH range of about 5.5 to about 6.5. The antimicrobial composition also maintains this antimicrobial efficacy and remains stable at a wide range of temperatures (about 25° C. to about 60° C.).

### EXAMPLE 1

### Preparation of Antimicrobial Composition

An example of the antimicrobial sanitizing composition was prepared.

First, 14 kg of deionized water at 100° C. was added to a 15 gallon, flat bottom container. Then, an additional 4.692 kg deionized water at 17° C. was added, to bring the final temperature to 51° C. The pH of the water was 6.24. Next, 120 g of a phenoxyethanol-based preservative (Nipaguard® IPP2, Clariant, Muttenz, Switzerland) was dissolved in the water. Next, 50 g carbomer (Carbopol® ETD 2623 or Carbopol® 980 NF, Noveon, Cleveland, Ohio) and 10 g xantham gum were mixed into the water solution and allowed to disperse for 40 minutes with continuous mixing. Next, 6 g of 0.15 N ACS was added to the solution and mixed for 15 minutes.

The anhydrous water soluble solvent complex was prepared by adding 30 g TEA, 600 g glycerin, and 400 g polypropylene glycol to a separate container while mixing and heating. The components were mixed until a homogeneous solution was obtained. Next, 20 g of PCMX (Nipacide™ MX, Clariant) was added to the container and mixed until completely dissolved.

A mixture of vitamin E and fragrance was prepared by adding 2 g of vitamin E into 40 g of fragrance in a separate, closed container and mixing until the components were dissolved. This mixture was then added to the water phase. Next, 5 g of aloe vera gel was also mixed into the water phase. Mixing continued for five minutes.

Finally, the PCMX solution was added to the water phase and mixed for 60 minutes. The final pH of the antimicrobial solution was 5.47. The components of the sanitizing composition were as follows:

| Component | W/W % |
|---|---|
| Deionized water | Up to 100% |
| Carbomer | 0.25 |
| Xantham gum | 0.05 |
| Preservative | 0.6 |
| PCMX | 0.1 |
| TEA | 0.15 |
| Propylene glycol | 2.0 |
| Glycerin | 3.0 |
| ACS 0.15 N | 0.03 |
| Vitamin E succinate | 0.01 |
| Fragrance | 0.2 |
| Aloe vera gel | 0.05 |

### EXAMPLE 2

### Antimicrobial Activity of Composition

Three samples of the antimicrobial composition prepared in Example 1 were taken, one each from the top (Sample 1), middle (Sample 2), and bottom (Sample 3) of the mixing container. To evaluate the antimicrobial efficacy of the composition, a time-kill study was conducted in accordance with the F.D.A.'s Tentative Final Monograph for Healthcare Antiseptic Drug Products (21 C.F.R. §333). The present study used common in vitro time-kill methodology, which demonstrates the speed of antimicrobial action within a time frame and the antimicrobial spectrum under controlled conditions. The time-kill kinetic study was performed with a select group of Gram positive and Gram negative microbial species. The four standard microorganism strains used in the study were: (1) Salmonella choleraesuis 6539, overnight culture, (2) Pseudomonous aeruginosa 9027, overnight culture, (3) Staphlococcus aureus 6538, overnight culture, and (4) E. coli O157 43894, overnight culture (ATCC, Manassas, Va.).

The CFU was calculated for each sample, and the results are shown in Table 1 below.

TABLE 1
Plate Count Data

| | S. Choleraesuis | P. Aeruginosa | S. Aureus | E. Coli |
|---|---|---|---|---|
| Sample | (CFU/mL) | (CFU/mL) | (CFU/mL) | (CFU/mL) |
| 1 | <6.67 | <6.67 | 8.73 × 10² | 8.67 × 10¹ |
| 2 | <6.67 | <6.67 | <6.67 | <6.67 |
| 3 | <6.67 | <6.67 | <6.67 | <6.67 |
| Control | 1.71 × 10⁶ | 1.13 × 10⁷ | 3.19 × 10⁶ | 9.07 × 10⁶ |

The plate-count data (CFU) were converted to log scale reduction and percent reduction, as shown below in Table 2. The log or percent reduction in microbial populations provided by an antimicrobial composition correlates to antimicrobial activity. After contact times generally ranging from 15 seconds to 5 minutes, a log reduction of 3-5 is most preferred, a log reduction of 1-3 is less preferred, and a log reduction of less than 1 is acceptable. A highly preferred antimicrobial composition will exhibit a 3-5 log reduction against a broad spectrum of microbial organisms in a short contact time.

TABLE 2
Log Scale and Percent Reduction

| | S. Choleraesuis | P. Aeruginosa | S. Aureus | E. Coli |
|---|---|---|---|---|
| Sample | Log/% | Log/% | Log/% | Log/% |
| 1 | >5.41/99.9996% | >6.23/99.9999% | >6.23/99.9999% | 5.02/99.9999% |
| 2 | >5.41/99.9996% | >6.23/99.9999% | >5.68/99.9998% | >6.13/99.9999% |
| 3 | >5.41/99.9996% | >6.23/99.9999% | >5.68/99.9998% | >6.13/99.9999% |

The results indicate that the antimicrobial composition demonstrates a high level of antimicrobial efficacy after a short contact time.

### EXAMPLE 3

### Preparation of Antimicrobial Composition without Preservative

An example of the antimicrobial composition was prepared which did not include preservative. The composition was prepared according to the procedure described in Example 1, except that the preservative was not added and 0.20 g of fragrance was added instead of 0.40 g.

The final pH of the antimicrobial composition was 5.4. The components of the antimicrobial composition were as follows:

| Component | W/W % |
|---|---|
| Deionized water | Up to 100% |
| Carbomer | 0.25 |
| Xantham gum | 0.05 |
| Preservative | 0 |
| PCMX | 0.1 |
| TEA | 0.15 |
| Propylene glycol | 2.0 |
| Glycerin | 3.0 |
| ACS 0.15 N | 0.03 |
| Vitamin E succinate | 0.01 |
| Fragrance | 0.1 |
| Aloe vera gel | 0.05 |

### EXAMPLE 4

### Antimicrobial Activity of Composition Prepared without Preservative

Three samples of the antimicrobial composition prepared in Example 3 were taken, one each from the top (Sample 1), middle (Sample 2), and bottom (Sample 3) of the mixing container. An in vitro time-kill study in accordance with the procedure described in Example 2 was performed. The CFU was calculated for each sample, and the results are shown in Table 3 below.

TABLE 3
Plate Count Data

| | S. Choleraesuis | P. Aeruginosa | S. Aureus |
|---|---|---|---|
| Sample | (CFU/mL) | (CFU/mL) | (CFU/mL) |
| 1 | <6.67 | <6.67 | 6.00 × 10¹ |
| 2 | <6.67 | 6.67 × 10⁰ | 5.33 × 10¹ |
| 3 | <6.67 | 9.33 × 10¹ | 8.00 × 10¹ |
| Control | 4.81 × 10⁶ | 1.35 × 10⁷ | 2.80 × 10⁶ |

The plate-count data (CFU) were converted to log scale reduction and percent reduction, as shown below in Table 4.

TABLE 4
Log Scale and Percent Reduction

| | S. Choleraesuis | P. Aeruginosa | S. Aureus |
|---|---|---|---|
| Sample | Log/% | Log/% | Log/% |
| 1 | >5.86/99.9999% | >6.31/99.9999% | >4.67/99.9979% |
| 2 | >5.86/99.9999% | >6.31/99.9999% | >4.72/99.9981% |
| 3 | >5.86/99.9999% | >5.16/99.9993% | >4.54/99.9971% |

The results indicate that the antimicrobial composition without preservative demonstrates a high level of antimicrobial efficacy after a short contact time.

### EXAMPLE 5

### Antimicrobial Activity of Composition Prepared without Active Ingredient

A composition was prepared in accordance with the procedure described in Example 1, except that the antimicrobial agent (PCMX) was not added. The final pH of the antimicrobial composition was 5.4. The components of the antimicrobial composition were as follows:

| Component | W/W % |
|---|---|
| Deionized water | Up to 100% |
| Carbomer | 0.25 |
| Xantham gum | 0.05 |
| Preservative | 0.5 |
| PCMX | 0 |
| TEA | 0.15 |
| Propylene glycol | 2.0 |
| Glycerin | 3.0 |
| ACS 0.15 N | 0.03 |
| Vitamin E succinate | 0.01 |
| Fragrance | 0.1 |
| Aloe vera gel | 0.05 |

An in vitro time-kill study in accordance with the procedure described in Example 2 was performed. The CFU was calculated for each sample, and the results are shown in Table 5 below.

TABLE 5
Plate Count Data

| | S. Choleraesuis | P. Aeruginosa | S. Aureus | E. Coli |
|---|---|---|---|---|
| | (CFU/mL) | (CFU/mL) | (CFU/mL) | (CFU/mL) |
| Antimicrobial | 6.80 × 10⁵ | 1.43 × 10⁶ | 2.67 × 10⁶ | 8.20 × 10⁶ |
| Composition | | | | |
| Control | 2.53 × 10⁶ | 1.54 × 10⁷ | 4.55 × 10⁶ | 1.35 × 10⁷ |

The plate-count data (CFU) were converted to log scale reduction and percent reduction, as shown below in Table 6.

TABLE 6
Log Scale and Percent Reduction

| | S. Choleraesuis | P. Aeruginosa | S. Aureus | E. Coli |
|---|---|---|---|---|
| | Log/% | Log/% | Log/% | Log/% |
| Prepared | 6.80 × 10⁵ | 1.43 × 10⁶ | 2.67 × 10⁶ | 8.20 × 10⁶ |
| Composition | | | | |

The results indicate that the active antimicrobial ingredient (PCMX) is required for the antimicrobial composition to work effectively.

### EXAMPLE 6

### Antimicrobial Activity of Compositions Prepared with Different pH Levels

Four samples of the antimicrobial composition were prepared, in accordance with the procedure described in Example 1 above, with variable amounts of TEA and having the compositions shown below:

| Component | W/W % |
|---|---|
| Deionized water | Up to 100% |
| Carbomer | 0.25 |
| Xantham gum | 0.05 |
| Preservative | 0.5 |
| PCMX | 0.3 |
| TEA: | |
| Sample 1 | 0.15 |
| Sample 2 | 0.186 |
| Sample 3 | 0.19 |
| Sample 4 | 0.24 |
| Propylene glycol | 2.0 |
| Glycerin | 3.0 |
| Vitamin E succinate | 0.01 |
| Fragrance | 0.1 |
| Aloe vera gel | 0.05 |

The samples had final pH values of 5.5 (Sample 1), 6.00 (Sample 2), 6.2 (Sample 3), and 6.4 (Sample 4). An in vitro time-kill study in accordance with the procedure described in Example 2 was performed. The CFU was calculated for each sample, and the results are shown in Table 7 below.

TABLE 7
Plate Count Data

| | | S. Choleraesuis | P. Aeruginosa | S. Aureus | E. Coli |
|---|---|---|---|---|---|
| Sample | pH | (CFU/mL) | (CFU/mL) | (CFU/mL) | (CFU/mL) |
| 1 | 5.5 | <6.67 | 3.93 × 10² | <6.67 | <6.67 |
| 2 | 6.0 | <6.67 | <6.67 | <6.67 | <6.67 |
| 3 | 6.2 | <6.67 | <6.67 | <6.67 | <6.67 |
| 4 | 6.4 | <6.67 | <6.67 | <6.67 | <6.67 |
| Control | 7.38 | 2.79 × 10⁶ | 7.47 × 10⁶ | 5.04 × 10⁶ | 1.43 × 10⁷ |

The plate-count data (CFU) were converted to log scale reduction and percent reduction, as shown below in Table 8.

TABLE 8
Log Scale and Percent Reduction

| | S. Choleraesuis | P. Aeruginosa | S. Aureus | E. Coli |
|---|---|---|---|---|
| Sample | Log/% | Log/% | Log/% | Log/% |
| 1 | >5.62/99.9999% | 4.28/99.9947% | >5.88/>99.9999% | >6.33/>99.9999% |
| 2 | >5.62/99.9999% | >6.04/>99.9999% | >5.88/>99.9999% | >6.33/>99.9999% |
| 3 | >5.62/99.9999% | >6.04/>99.9999% | >5.88/>99.9999% | >6.33/>99.9999% |
| 4 | >5.62/99.9999% | >6.04/>99.9999% | >5.88/>99.9999% | >6.33/>99.9999% |

The results indicate that the antimicrobial composition demonstrates a high antimicrobial efficacy throughout the pH range of 5.5 to 6.5.

### EXAMPLE 7

### Antimicrobial Activity of Composition at Elevated Temperature

The four samples of antimicrobial composition prepared in Example 6 were stored at 60° C. for 2.5 weeks. An in vitro time-kill study in accordance with the procedure described in Example 2 was performed. The CFU was calculated for each sample and results are shown in Table 9 below.

TABLE 9
Plate Count Data

| | | S. Choleraesuis | P. Aeruginosa | S. Aureus | E. Coli |
|---|---|---|---|---|---|
| Sample | pH | (CFU/mL) | (CFU/mL) | (CFU/mL) | (CFU/mL) |
| 1 | 5.5 | <6.67 | <6.67 | 1.03 × 10⁴ | <6.67 |
| 2 | 6.0 | <6.67 | <6.67 | <6.67 | <6.67 |
| 3 | 6.2 | <6.67 | <6.67 | <6.67 | <6.67 |
| 4 | 6.4 | <6.67 | <6.67 | <6.67 | <6.67 |
| Control | 7.38 | 2.15 × 10⁶ | 1.49 × 10⁷ | 3.03 × 10⁶ | 1.40 × 10⁷ |

The plate-count data (CFU) were converted to log scale reduction and percent reduction, as shown below in Table 10.

TABLE 10
Log Scale and Percent Reduction

| | | P. Aeruginosa | S. Aureus | |
|---|---|---|---|---|
| Sample | S. Choleraesuis Log/% | Log/% | Log/% | E. Coli Log/% |
| 1 | 5.51/>99.9997% | 6.35/>99.9999% | 2.48/99.6601% | 6.32/99.9999% |
| 2 | 5.51/>99.9997% | 6.35/>99.9999% | 5.66/99.9998% | 6.32/99.9999% |
| 3 | 5.51/>99.9997% | 6.35/>99.9999% | 5.66/99.9998% | 6.32/99.9999% |
| 4 | 5.51/>99.9997% | 6.35/>99.9999% | 5.66/99.9998% | 6.32/99.9999% |

The results indicate that the antimicrobial composition demonstrates a high level of antimicrobial efficacy and is stable at a wide range of temperatures (25° C.-60° C.).

### EXAMPLE 8

### Comparative Study

A sample of the Antimicrobial Sanitizing Composition of one embodiment of the present invention was compared to five commercially available antimicrobial compositions. The five comparison products were each classified as over-the-counter general-use antimicrobial handwashing products, particularly for use by healthcare personnel, which contain PCMX as the active ingredient. At the time of the study, no water-based products using PCMX were available on the market, so the comparison products are in the form of emulsion creams, soaps, and alcohol-based gels. The products used in the study are shown in Table 11 below.

TABLE 11
Products Used in Comparative Study

| Product | Appearance | Active Ingredient | pH |
|---|---|---|---|
| Antimicrobial Sanitizing | Water-based gel | PCMX, 0.3% | 5.6 |
| Composition | | | |
| Microcyde ™ Lotion | Emulsion cream | PCMX, 1.0% | 7.2 |
| (Loring Sarsfield, Newburyport, MA) | | | |
| Microcyde ™ Skin Cleaner | Soap | PCMX, 1.8% | 6.5 |
| (Loring Sarsfield, Newburyport, MA) | | | |
| PROVON ® Healthcare Personnel | Soap | PCMX, 0.5% | 5.8-6.2 |
| Handwash | | | |
| (GOJO Industries, Akron, Ohio) | | | |
| PROVON ® Medicated Lotion Soap | Soap | PCMX, 0.3% | 8.6-9.2 |
| (GOJO Industries, Akron, Ohio) | | | |
| PURELL ® Instant Hand Sanitizer | Alcohol-Based | Ethyl alcohol, 62% | 7.5-8.5 |
| (GOJO Industries, Akron, Ohio) | Gel | | |

An in vitro time-kill study using a contact time of 30 seconds was performed as described in Example 2. The CFU was calculated for each sample and results are shown in Table 12 below.

TABLE 12
Plate Count Data

| | S. Choleraesuis | P. Aeruginosa | S. Aureus | E. Coli |
|---|---|---|---|---|
| Product | (CFU/mL) | (CFU/mL) | (CFU/mL) | (CFU/mL) |
| Antimicrobial | <6.67 | <6.67 | 1.00 × 10² | <6.67 |
| Sanitizing | | | | |
| Composition | | | | |
| Microcyde ™ Lotion | 1.47 × 10⁶ | 8.53 × 10⁴ | 4.73 × 10⁶ | 1.64 × 10⁷ |
| Microcyde ™ Skin | <6.67 | <6.67 | 6.13 × 10³ | <6.67 |
| Cleaner | | | | |
| PROVON ® | <6.67 | 2.73 × 10² | 1.75 × 10⁶ | 4.57 × 10⁶ |
| Healthcare Personnel | | | | |
| Handwash | | | | |
| PROVON ® | <6.67 | <6.67 | 1.09 × 10⁶ | 1.02 × 10⁶ |
| Medicated Lotion | | | | |
| Soap | | | | |
| PURELL ® Instant | <6.67 | <6.67 | <6.67 | <6.67 |
| Hand Sanitizer | | | | |
| Control | 1.94 × 10⁶ | 8.47 × 10⁶ | 4.48 × 10⁶ | 1.43 × 10⁷ |
| (pH7.38 phosphate buffer) | | | | |

The plate-count data (CFU) were converted to log scale reduction and percent reduction, as shown below in Table 13.

TABLE 13
Log Scale and Percent Reduction

| | S. Choleraesuis | P. Aeruginosa | S. Aureus | E. Coli |
|---|---|---|---|---|
| Product | (CFU/mL) | (CFU/mL) | (CFU/mL) | (CFU/mL) |
| Antimicrobial | >5.46/>99.9997% | >6.10/99.9999% | 4.65/99.9978% | >6.33/99.9999% |
| Sanitizing | | | | |
| Composition | | | | |
| Microcyde ™ | 0.12/24.2268% | 1.99/98.9929% | 0/0 | 0/0 |
| Lotion | | | | |
| Microcyde ™ | >5.46/>99.9997% | >6.10/99.9999% | 2.86/99.8632% | >6.33/99.9999% |
| Skin Cleaner | | | | |
| PROVON ® | >5.46/>99.9997% | 4.49/99.9968% | 0.41/60.9375% | 0.49/68.0419% |
| Healthcare | | | | |
| Personnel | | | | |
| Handwash | | | | |
| PROVON ® | >5.46/>99.9997% | >6.10/99.9999% | 0.61/75.6696% | 1.15/92.8671% |
| Medicated | | | | |
| Lotion Soap | | | | |
| PURELL ® | >5.46/>99.9997% | >6.10/99.9999% | >5.83/99.9999% | >6.33/99.9999% |
| Instant Hand | | | | |
| Sanitizer | | | | |

While all six products showed an ability to reduce at least some microorganism loads in a short contact time, the current antimicrobial sanitizing solution and the PURELL® Instant Hand Sanitizer (which was an alcohol-based gel) showed the best results. In some instances, the other four products did not achieve the log reduction achieved by the antimicrobial sanitizing composition, nor did they achieve the preferred log reduction amounts of 3-5 or 1-3.

1. A surfactant-free, alcohol-free, water based antimicrobial composition comprising from about 0.1% to about 3.75% by weight of a dissolved phenolic antimicrobial agent.

2. The antimicrobial composition of claim 1, wherein the dissolved phenolic antimicrobial agent is chloroxylenol (“PCMX”), 2,4,4′-trichloro-2′-hydroxy-diphenylether, benzylalkonium chloride, or 4-chloro-3,5-dimethylphenol.

3. The antimicrobial composition of claim 1, further comprising a glycol, glycerin, a neutralizing agent, a preservative, an acrylic polymer, a polysaccharide polymer, and water.

4. The antimicrobial composition of claim 1, further comprising a fragrance, an additional moisturizer, and a pH adjuster.

5. A surfactant-free, alcohol-free, water based antimicrobial composition comprising, by weight:
(a) From about 0.1% to about 3.75% of a dissolved phenolic antimicrobial agent;
(b) From about 1% to about 8% of a glycol;
(c) From about 2% to about 10% of glycerin;
(d) From about 0.1% to about 1% of a neutralizing agent;
(e) From about 0.1% to about 1% of a preservative;
(f) From about 0.1% to about 1% of an acrylic polymer;
(g) From about 0.02% to about 0.5% of a polysaccharide polymer; and
(h) Remainder water.

6. The antimicrobial composition of claim 5, wherein the dissolved phenolic antimicrobial agent is 2,4,4′-trichloro-2′-hydroxy-diphenylether, benzylalkonium chloride, or 4-chloro-3,5-dimethylphenol.

7. The antimicrobial composition of claim 5, wherein the dissolved phenolic antimicrobial agent is chloroxylenol (“PCMX”).

8. The antimicrobial composition of claim 5, wherein the glycol is hexylene glycol, triethylene glycol, ethylene glycol, or diethylene glycol.

9. The antimicrobial composition of claim 5, wherein the glycol is propylene glycol.

10. The antimicrobial composition of claim 5, wherein the neutralizing agent is sodium hydroxide, potassium hydroxide, ammonium hydroxide, a polar organic amine, a low polarity amine, or an amino acid.

11. The antimicrobial composition of claim 5, wherein the neutralizing agent is triethanolamine (“TEA”).

12. The antimicrobial composition of claim 5, wherein the preservative is chlorphenesin, iodopropynyl butylcarbamate, benzoic acid, potassium sorbate, or sorbic acid.

13. The antimicrobial composition of claim 5, wherein the preservative is phenoxyethanol.

14. The antimicrobial composition of claim 13, further comprising iodine carbamate.

15. The antimicrobial composition of claim 5, wherein the acrylic polymer is a carbomer.

16. The antimicrobial composition of claim 5, wherein the polysaccharide polymer is methylcellulose, propylcellulose, acacia gum, arabic gum, gum ghatti, guar gum, benzoin gum, tamarind gum, karaya gum, or gum accroides.

17. The antimicrobial composition of claim 5, wherein the polysaccharide polymer is xantham gum.

18. The antimicrobial composition of claim 5, further comprising from about 0.05% to about 0.5% by weight of a fragrance.

19. The antimicrobial composition of claim 5, further comprising an additional moisturizer, wherein the additional moisturizer is vitamin E, aloe vera, a polyol, or a mixture thereof.

20. The antimicrobial composition of claim 19, wherein the additional moisturizer comprises from about 0.005% to about 0.4% by weight of vitamin E.

21. The antimicrobial composition of claim 20, wherein the vitamin E is vitamin E succinate or vitamin E acetate.

22. The antimicrobial composition of claim 19, wherein the additional moisturizer comprises from about 0.025% to about 1.0% by weight of aloe vera.

23. The antimicrobial composition of claim 19, wherein the polyol is glycerin, sorbitol, mannitol, maltitol, isomalt, xylitol, erythritol, or a mixture thereof.

24. The antimicrobial composition of claim 5, further comprising a pH adjuster.

25. The antimicrobial composition of claim 24, wherein the pH adjuster is an organic acid or an inorganic acid.

26. The antimicrobial composition of claim 24, wherein the pH adjuster comprises from about 0.01% to about 0.05% by weight of acidified calcium sulfate (“ACS”).

27. A surfactant-free, alcohol-free, water based antimicrobial composition comprising, by weight:
(a) From about 0.1% to about 0.75% of a dissolved phenolic antimicrobial agent;
(b) From about 1% to about 5% of a glycol;
(c) From about 2% to about 7% of glycerin;
(d) From about 0.1% to about 0.5% of a neutralizing agent;
(e) From about 0.3% to about 0.7% of a preservative;
(f) From about 0.1% to about 0.5% of an acrylic polymer;
(g) From about 0.02% to about 0.25% of a polysaccharide polymer; and
(h) Remainder water.

28. The antimicrobial composition of claim 27, wherein the dissolved phenolic antimicrobial agent is chloroxylenol (“PCMX”), 2,4,4′-trichloro-2′-hydroxy-diphenylether, benzylalkonium chloride, or 4-chloro-3,5-dimethylphenol.

29. The antimicrobial composition of claim 27, wherein the glycol is propylene glycol, hexylene glycol, triethylene glycol, ethylene glycol, or diethylene glycol.

30. The antimicrobial composition of claim 27, wherein the neutralizing agent is triethanolamine (“TEA”), sodium hydroxide, potassium hydroxide, ammonium hydroxide, a polar organic amine, a low polarity amine, or an amino acid.

31. The antimicrobial composition of claim 27, wherein the preservative is phenoxyethanol, chlorphenesin, iodopropynyl butylcarbamate, benzoic acid, potassium sorbate, or sorbic acid.

32. The antimicrobial composition of claim 31, further comprising iodine carbamate.

33. The antimicrobial composition of claim 27, wherein the acrylic polymer is a carbomer.

34. The antimicrobial composition of claim 27, wherein the polysaccharide polymer is xantham gum, methylcellulose, propylcellulose, acacia gum, arabic gum, gum ghatti, guar gum, benzoin gum, tamarind gum, karaya gum, or gum accroides.

35. The antimicrobial composition of claim 27, further comprising from about 0.05% to about 0.2% by weight of a fragrance.

36. The antimicrobial composition of claim 27, further comprising an additional moisturizer, wherein the additional moisturizer comprises from about 0.005% to about 0.2% by weight of vitamin E or vitamin E succinate.

37. The antimicrobial composition of claim 27, further comprising an additional moisturizer, wherein the additional moisturizer comprises from about 0.025% to about 0.5% by weight of an aloe vera gel.

38. The antimicrobial composition of claim 27, further comprising a pH adjuster, wherein the pH adjuster comprises from about 0.02% to about 0.04% by weight of acidified calcium sulfate (“ACS”).

39. A surfactant-free, alcohol-free, water based antimicrobial composition comprising, by weight:
(a) From about 0.1% to about 0.5% of a dissolved phenolic antimicrobial agent;
(b) From about 1% to about 4% of a glycol;
(c) From about 2% to about 5% of glycerin;
(d) From about 0.1% to about 0.4% of a neutralizing agent;
(e) From about 0.45% to about 0.6% of a preservative;
(f) From about 0.1% to about 0.3% of an acrylic polymer;
(g) From about 0.02% to about 0.075% of a polysaccharide polymer; and
(h) Remainder water.

40. The antimicrobial composition of claim 39, wherein the dissolved phenolic antimicrobial agent is chloroxylenol (“PCMX”), 2,4,4′-trichloro-2′-hydroxy-diphenylether, benzylalkonium chloride, or 4-chloro-3,5-dimethylphenol

41. The antimicrobial composition of claim 39, wherein the glycol is propylene glycol, hexylene glycol, triethylene glycol, ethylene glycol, or diethylene glycol.

42. The antimicrobial composition of claim 39, wherein the neutralizing agent is triethanolamine (“TEA”), sodium hydroxide, potassium hydroxide, ammonium hydroxide, a polar organic amine, a low polarity amine, or an amino acid.

43. The antimicrobial composition of claim 39, wherein the preservative is phenoxyethanol, chlorphenesin, iodopropynyl butylcarbamate, benzoic acid, potassium sorbate, or sorbic acid.

44. The antimicrobial composition of claim 43, further comprising iodine carbamate.

45. The antimicrobial composition of claim 39, wherein the acrylic polymer is a carbomer.

46. The antimicrobial composition of claim 39, wherein the polysaccharide polymer is xantham gum, methylcellulose, propylcellulose, acacia gum, arabic gum, gum ghatti, guar gum, benzoin gum, tamarind gum, karaya gum, or gum accroides.

47. The antimicrobial composition of claim 39, further comprising from about 0.05% to about 0.1% by weight of a fragrance.

48. The antimicrobial composition of claim 39, further comprising an additional moisturizer, wherein the additional moisturizer comprises from about 0.005% to about 0.07% of vitamin E or vitamin E succinate.

49. The antimicrobial composition of claim 39, further comprising an additional moisturizer, wherein the additional moisturizer comprises from about 0.025% to about 0.1% by weight of aloe vera gel.

50. The antimicrobial composition of claim 39, further comprising a pH adjuster, wherein the pH adjuster comprises about 0.03% by weight of acidified calcium sulfate (“ACS”).

51. An antimicrobial composition having a pH from about 5.5 to about 6.5 and comprising dissolved chloroxylenol (“PCMX”), propylene glycol, glycerin, triethanolamine (“TEA”), phenoxyethanol, carbomer, xantham gum, and deionized water.

52. The antimicrobial composition of claim 51, further comprising fragrance, vitamin E succinate, aloe vera gel, and acidified calcium sulfate (“ACS”).

53. A surfactant-free, alcohol-free, water based antimicrobial composition comprising, by weight:
(a) About 0.3% of dissolved chloroxylenol (“PCMX”);
(b) About 2% of propylene glycol;
(c) About 3% of glycerin;
(d) About 0.15% of triethanolamine (“TEA”);
(e) About 0.5% of phenoxyethanol;
(f) About 0.25% of carbomer;
(f) About 0.05% of xantham gum;
(g) About 0.10% of fragrance;
(h) About 0.01% of vitamin E succinate;
(i) About 0.05% of aloe vera gel; and
(j) Remainder water.

54. A method for preparing a surfactant-free, alcohol-free water based antimicrobial composition comprising a dissolved phenolic antimicrobial agent, comprising:
(a) combining water, a preservative, an acrylic polymer, and a polysaccharide polymer in a first container to form a polymer dispersion;
(b) combining a glycol, glycerin, and a neutralizing agent in a second container to form a solvent mixture;
(c) adding a phenolic antimicrobial agent to the solvent mixture in the second container to form a dissolved phenol mixture; and
(d) adding the dissolved phenol mixture to the polymer dispersion in the first container.

55. The method of claim 54, wherein said first combining step further comprises combining fragrance, additional moisturizers, and a pH adjuster to form the polymer dispersion.

56. A method for preparing a dissolved phenolic antimicrobial agent, comprising:
(a) combining a glycol, glycerin, and a neutralizing agent to form a solvent mixture; and
(b) adding a phenolic antimicrobial agent to the solvent mixture to give a dissolved phenolic antimicrobial agent.

57. The method of claim 56, further comprising heating the phenolic antimicrobial agent and the solvent mixture.

58. The method of claim 56, wherein the phenolic antimicrobial agent is PCMX.

59. The method of claim 56, wherein the glycol is propylene glycol.

60. The method of claim 56, wherein the neutralizing agent is TEA.

61. A method for reducing the number of microbial organisms on a surface, comprising:
contacting the surface with the antimicrobial composition of claim 5.

62. The method of claim 61, wherein the antimicrobial composition contacts the surface for about 30 or more seconds to achieve a log reduction of about 5 to about 6 against Gram positive or Gram negative bacteria.

63. A method for inhibiting the growth of microbial organisms on a surface, comprising:
contacting the surface with the antimicrobial composition of claim 5.
